(19) United States
(12) Patent Application Publication
(10) Pub. No.: US 2026/0166713 A1
(43) Pub. Date: Jun. 18, 2026

(54) WEARABLE ROBOT

(71) Applicants: Hyundai Motor Company, Seoul (KR); Kia Corporation, Seoul (KR)

(72) Inventors: Neung Hwan Yim, Hwaseong-si (KR); Sung Woo Park, Hwaseong-si (KR); Hyun Seop Lim, Hwaseong-si (KR)

(21) Appl. No.: 19/217,150

(22) Filed: May 23, 2025

(30) Foreign Application Priority Data
Dec. 17, 2024 (KR) 10-2024-0189166

(51) Int. Cl. B25J 9/00; A61F 5/01

(52) U.S. Cl. CPC B25J 9/0006; A61F 5/0111

(57) ABSTRACT

A wearable robot can include a connection part in which a lower leg of a human body of a user can be accommodated, a mechanism part including a five-joint rotation link part connected to the connection part and an elastic portion that is connected to a partial rotation center of the rotation link part and is configured to provide an elastic force to the connection part, and a support part connected to the mechanism part by a rotation joint, such that in a process of synchronizing an ankle joint and the mechanism part with each other by the connection part, one degree of freedom of underactuation among two degrees of freedom of the mechanism part can be offset and misalignment between the ankle joint and the mechanism part can be self-aligned.

### CROSS-REFERENCE TO RELATED APPLICATIONS

This application claims the benefit of priority to Korean Patent Application No. 10-2024-0189166, filed in the Korean Intellectual Property Office on Dec. 17, 2024, the entire contents of which are incorporated herein by reference.

### TECHNICAL FIELD

The present disclosure relates to a wearable robot.

### BACKGROUND

Generally, an ankle foot orthosis (AFO) of a patient has a shape based on a rigid body such as a cast and serves to assist food drop due to hemiplegia and walking exercise.

However, an ankle assistance device completely restricts actual movement of an ankle and thus has limitations in terms of rehabilitation.

A prior device may provide for adjusting a tensile force according to an age and a joint condition of a patient together with a function of adding the tensile force capable of preventing foot drop and lack of propulsion at the same time. However, in such prior device, movement of a body joint is divided into two parts (a wearing part and a support part) and thus may not be completely simulated, and there are limitations in using such device in various environments such as stairs and obstacles.

Another prior device has an advantage in that movement of a joint may be assisted according to patient walking through a control algorithm. However, due to an actuator in such prior device, it may be difficult to apply the ankle assistance device in real life due to lack of aesthetics and wearability due to a large volume and a large weight together with price competitiveness. In addition, there may be a limitation in that the actuator should be positioned close to an actual ankle in order to minimize misalignment occurring between the actuator and the joint due to different dimensions of living joints for each person. Thus, there are numerous problems of the prior devices.
Korean Patent No. KR 1682450 (2016 Nov. 29) and
U.S. Pat. No. 8,075,633 (2011 Dec. 13) disclose information related to information disclosed herein.

### SUMMARY

The present disclosure relates to a wearable robot applied to an ankle assistance device and capable of self-alignment.

An embodiment of the present disclosure can solve the above-mentioned problems occurring in the prior art while advantages achieved by the prior art can be maintained intact.

An embodiment of the present disclosure can provide a wearable robot capable of inducing natural walking by securing a degree of freedom allowing an ankle joint to freely move.

An embodiment of the present disclosure can provide a wearable robot capable of assisting non-linear body joint movement by adding a mechanical part including a rotation link part and an elastic portion as well as a connection part and a support part.

An embodiment of the present disclosure can provide a wearable robot capable of promoting not only wearability but also versatility and modularity between users by minimizing a volume and a weight of a device, providing a self-alignment function to a mechanism itself, and positioning a mechanical part to be spaced apart from an ankle.

An embodiment of the present disclosure can provide a wearable robot that may be customized and thus convenient and may assist bidirectional movement of an ankle joint of a human body, such as foot drop or ground pushing-off, during walking.

Technical problems that can be solved by an embodiment of the present disclosure are not necessarily limited to the aforementioned problems, and solutions to other technical problems not mentioned herein using an embodiment of the present disclosure can be understood from the following description by those skilled in the art to which the present disclosure pertains.

According to an embodiment of the present disclosure, a wearable robot can include a connection part in which a lower leg of a human body of a user is accommodated, a mechanism part including a five-joint rotation link part connected to the connection part and an elastic portion that is connected to a partial rotation center of the rotation link part and provides an elastic force to the connection part, and a support part connected to the mechanism part by a rotation joint, wherein, in a process of synchronizing an ankle joint and the mechanism part with each other by the connection part, one degree of freedom of underactuation among two degrees of freedom of the mechanism part is offset, and thus misalignment between the ankle joint and the mechanism part can be self-aligned.

According to an embodiment of the present disclosure, the rotation link part may include a first rotation link connected to the support part by a first rotation joint, a second rotation link connected to the first rotation link by a second rotation joint, a third rotation link connected to the second rotation link by a third rotation joint, a fourth rotation link connected to the first rotation link by a fourth rotation joint, and a fifth rotation link connected to the third rotation link by a fifth rotation joint.

According to an embodiment of the present disclosure, the fifth rotation link may be connected to the fourth rotation link by a sixth rotation joint.

According to an embodiment of the present disclosure, the first rotation link may be connected to the support part by the first rotation joint, may be connected to the second rotation link by the second rotation joint, and may be connected to the fourth rotation link by the fourth rotation joint.

According to an embodiment of the present disclosure, the elastic portion may include a first elastic portion that is elastically supported by the first rotation joint of the first rotation link and rotates the first rotation link to a front side about the first rotation joint, a second elastic portion that is elastically supported by the second rotation joint of the first rotation link and rotates the second rotation link to the front side about the second rotation joint, and a third elastic portion that is elastically supported by the third rotation joint of the third rotation link and rotates the third rotation link to the front side about the third rotation joint.

According to an embodiment of the present disclosure, the first rotation link may be installed to support the support part in one degree of freedom of rotation and may be positioned behind the ankle joint.

According to an embodiment of the present disclosure, the second rotation link may be positioned behind the first rotation link, and the fourth rotation link and the fifth rotation link may be positioned in front of the first rotation link.

According to an embodiment of the present disclosure, the third rotation joint connecting the second rotation link and the third rotation link may be installed at an uppermost one of the rotation links.

According to an embodiment of the present disclosure, the sixth rotation joint may be positioned below the fourth rotation joint by which the first rotation link and the fourth rotation link are connected to each other.

According to an embodiment of the present disclosure, the fourth rotation link may correspond to face the third rotation link and may be positioned below the third rotation link.

According to an embodiment of the present disclosure, the second elastic portion and the third elastic portion may have a maximum deformation angle within a range of +10°.

According to an embodiment of the present disclosure, the connection part may be positioned to correspond to the lower leg of the user and may be connected to the mechanism part by the fifth rotation link.

According to an embodiment of the present disclosure, the support part may include a support frame supported by a sole of the user and a support link connected between the first rotation link of the mechanism part and the support frame.

### BRIEF DESCRIPTION OF THE DRAWINGS

The above and other features and advantages of example embodiments of the present disclosure can be more apparent from the following detailed description taken in conjunction with the accompanying drawings, in which:

FIG. 1 is a side view illustrating a wearable robot according to an embodiment of the present disclosure;

FIG. 2 is an enlarged side view illustrating a mechanism part of a wearable robot according to an embodiment of the present disclosure;

FIG. 3 is a kinematic schematic illustrating a state in which a wearable robot and an ankle joint of a human body are synchronized with each other through one rotation link part according to an embodiment of the present disclosure;

FIG. 4 is a side view illustrating an operation state in a state in which there is no foot drop of a wearable robot according to an embodiment of the present disclosure; and

FIG. 5 is a side view illustrating an operation state in a state in which a wearable robot is in a ground pushing-off state according to an embodiment of the present disclosure.

### DETAILED DESCRIPTION OF ILLUSTRATIVE EMBODIMENTS

Hereinafter, some example embodiments of the present disclosure will be described in detail with reference to the drawings. When reference numerals are added to the components of the drawings, the same components can have the same reference numerals as much as possible even when the components are illustrated in different drawings. Further, in describing the example embodiments of the present disclosure, a detailed description of the related known configuration or function can be omitted when it is determined that it might interfere with the understanding of the embodiment of the present disclosure.

In the description of components of the embodiments of the present disclosure, terms such as “first,” “second,” “A”, “B”, “(a)”, and “(b)” may be used. Such terms can be merely intended to distinguish one component from other components, and such terms do not necessarily limit the nature, order, or sequence of the components. It can be understood that when one component is “connected”, “coupled” or “joined” to another component, the former may be directly connected or jointed to the latter or may be “connected”, “coupled” or “joined” to the latter with a third component interposed therebetween.

Hereinafter, a wearable robot according to an example embodiment of the present disclosure will be described in detail with reference to the accompanying drawings.

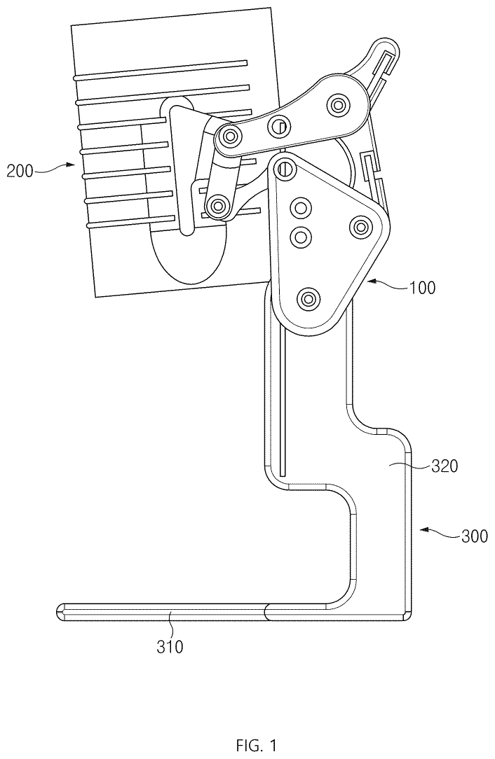

FIG. 1 is a side view illustrating a wearable robot according to an embodiment of the present disclosure.

As illustrated in FIG. 1, a wearable robot according to an embodiment of the present disclosure may include a mechanism part 100, a connection part 200, and a support part 300.

The wearable robot may be formed to simulate an ankle joint of a human body of a user and may include a rotation link structure having optimum conformity to simulate nonlinear movement of the ankle joint.

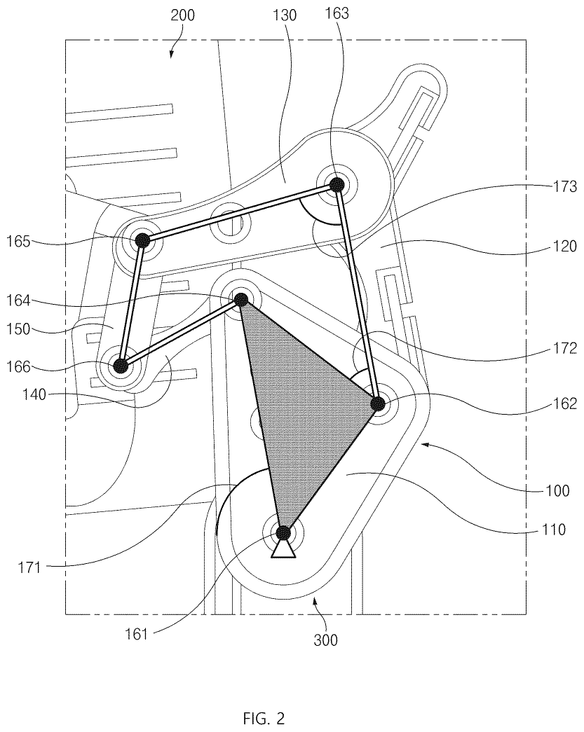

FIG. 2 is an enlarged side view illustrating the mechanism part 100 of a wearable robot according to an embodiment of the present disclosure.

As illustrated in FIG. 2, the mechanism part 100 may include rotation link parts 110 to 150 formed to be rotatable and elastic portions 171 to 173 that can assist movement between a portion of the human body and the mechanism part 100 in a specific rotation direction.

The mechanism part 100 may be located above the support part 300. The mechanism part 100 may include the first rotation link 110 connected to the support part 300 by a first rotation joint 161.

The first rotation link 110 may be a connection arm installed to support the support part 300 at one degree of freedom of rotation. The first rotation link 110 may be located behind the ankle joint of the user.

The mechanism part 100 may include the second rotation link 120 connected to the first rotation link 110 by a second rotation joint 162. The second rotation link 120 may be maintained in a state of being approximately vertical upward from the first rotation link 110.

The mechanism part 100 may include the third rotation link 130 connected to the second rotation link 120 by a third rotation joint 163. The third rotation link 130 may be maintained in a state of being approximately parallel forward from the second rotation link 120.

In this example, the third rotation joint 163 connects to the second rotation link 120 and the third rotation link 130 may be installed at an uppermost position of the rotation links. Due to this configuration, an overlapping phenomenon between the rotation link parts 110 to 150 may be minimized.

The mechanism part 100 may include a fourth rotation link 140 connected to the first rotation link 110 by a fourth rotation joint 164. The fourth rotation link 140 may correspond to face the third rotation link 130 and may be located below the third rotation link 130.

The mechanism part 100 may include a fifth rotation link 150 connected to the third rotation link 130 by a fifth rotation joint 165. The fifth rotation link 150 may be connected to the fourth rotation link 140 by a sixth rotation joint 166.

The fifth rotation link 150 may connect the connection part 200 and the mechanism part 100 and may be connected by the sixth rotation joint 166.

In this example, the second rotation link 120 may be located behind the first rotation link 110, and the fourth rotation link 140 and the fifth rotation link 150 may be located in front of the first rotation link 110.

The sixth rotation joint 166 by which the fourth rotation link 140 and the fifth rotation link 150 are connected to each other may be located below the fourth rotation joint 164 by which the first rotation link 110 and the fourth rotation link 140 are connected to each other. Due to this configuration, a bifurcation phenomenon due to nonlinearity of the rotation link parts 110 to 150 may be minimized.

The first rotation link 110 may be connected to the support part 300 by the first rotation joint 161, connected to the second rotation link 120 by the second rotation joint 162, and connected to the fourth rotation link 140 by the fourth rotation joint 164.

The first rotation joint 161 may include the first elastic portion 171.

The first elastic portion 171 may generate a moment to assist the ankle joint of the human body through elastic energy. The first elastic portion 171 may elastically support the first rotation link 110 toward the front side about the first rotation joint 161. The first elastic portion 171 may store elastic energy according to a change in an inclination of a lower leg of the user and provide a driving force to the lower leg of the user when the user walks.

The second rotation joint 162 may include the second elastic portion 172, and the third rotation joint 163 may include the third elastic portion 173.

The second elastic portion 172 may adjust alignment between the ankle joint of the human body and the mechanism part 100. That is, to minimize misalignment between the ankle joint of the human body of the user and the mechanism part 100, the second elastic portion 172 may elastically support the second rotation link 120 to the front side about to the second rotation joint 162.

Like the second elastic portion 172, the third elastic portion 173 may also adjust the alignment between the ankle joint of the human body and the mechanism part 100. That is, the third elastic portion 173 may elastically support the third rotation link 130 toward the front side about the third rotation joint 163.

In this way, the elastic portions 171 to 173 may be located behind the ankle joint of the human body and elastically support the connection part 200 to the front side, and thus self-alignment may be performed.

In this way, as the mechanism part 100 may perform a self-alignment function, the mechanism part 100 may be positioned to be spaced apart from the ankle joint of the human body. Accordingly, device wearability as well as versatility between users and modularity may be achieved.

The second elastic portion 172 and the third elastic portion 173 may be configured such that a maximum deformation angle does not exceed ±10°.

The elastic portions 171 to 173 may be torsion springs and may be replaced with tension springs, compression springs, elastic bars, and the like.

The connection part 200 may be designed in consideration of wearability of the user and may connect the ankle joint of the human body of the user and the mechanism part 100.

The connection part 200 may be positioned to correspond to the lower leg of the user and may be connected between the portion (lower leg) of the human body of the user and the fifth rotation link 150 of the mechanism part 100. In the connection part 200, one degree of freedom of underactuation may be limited in a process in which the nonlinear movement of the ankle joint of the human body of the user and the movement of the mechanism part 100 coincide with each other.

The connection part 200 may have a cylindrical shape so that the lower leg of the human body of the user may be accommodated therein. The connection part 200 may be positioned to be spaced apart from the ankle joint of the human body together with the mechanism part 100.

The connection part 200 may adopt various shapes in consideration of a size of the human body of the user.

The support part 300 may be a part that can support the movement of a portion (sole) of the human body of the user and the mechanism part 100 and may be connected to the first rotation link 110 by the first rotation joint 161.

The support part 300 may include a support frame 310 positioned to correspond to a lower skeleton of the user, capable of rotating in multiple degrees of freedom, and capable of supporting the sole of the user and a support link 320 can be connected between the first rotation link 110 of the mechanism part 100 and the support frame 310.

The support link 320 may be formed integrally with the support frame 310, and may be rotatably connected to the first rotation link 110 of the mechanism part 100 by the first rotation joint 161. A translational movement of the support link 320 of the support part 300 may be limited by mechanical restraint of the rotation link parts 110 to 150 of the mechanism part 100.

Like the connection part 200, the support part 300 may be configured in consideration of the size of the human body of the user.

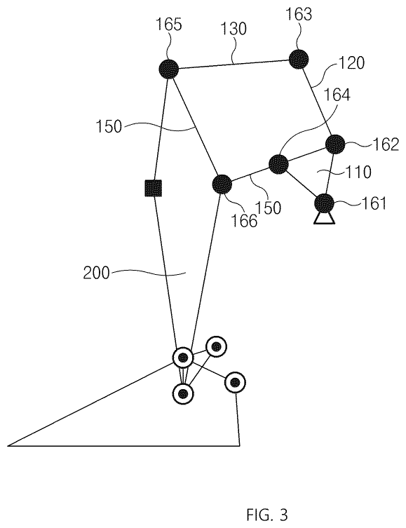

FIG. 3 is a kinematic schematic illustrating a state in which the wearable robot and an ankle joint of a human body are synchronized with each other through one rotation link part according to an embodiment of the present disclosure.

Referring to FIGS. 1 to 3, the mechanism part 100 can include a two-degree-of-freedom underactuated mechanism including the first to fifth rotation link parts 110 to 150 and the first to sixth rotation joints 161 to 166. That is, a wearable robot according to an embodiment of the present disclosure may implement a two-degree-of-freedom underactuated mechanism including the five rotation link parts 110 to 150 and the six rotation joints 161 to 166.

The ankle joint of the human body may be implemented by a nonlinear one-degree-of-freedom four-joint linkage mechanism.

Thus, when the user wears the wearable robot, the one degree of freedom of the underactuation among the two degrees of freedom of the mechanism part 100 may be implemented as a one-degree-of-freedom eight-joint full actuated linkage mechanism as the ankle joint of the human body and the rotation link parts 110 to 150 of the mechanism part 100 are synchronized with each other.

In this way, the wearable robot that may be driven by the one-degree-of-freedom eight-joint full actuated linkage mechanism may perform self-alignment.

The ankle joint of the human body and the fifth rotation link 150 among the rotation link parts 110 to 150 of the mechanism part 100 may be synchronized to one rotation link through the connection part 200. That is, before the wearing, the wearable robot according to an embodiment of the present disclosure may be a two-degree-of-freedom underactuated mechanism. However, when the wearable robot is worn on the ankle joint of the human body, the ankle joint of the human body and the fifth rotation link 150 of the mechanism part 100 may be synchronized with each other, the one degree of freedom of the underactuation among the two degrees of freedom of the mechanism part 100 may be offset, and thus the one-degree-of-freedom full actuated mechanism may be implemented.

The rotation link parts 110 to 150 included in the mechanism part 100 of the wearable robot according to an embodiment of the present disclosure may secure the following characteristics to perform the self-alignment.

In an embodiment of the present disclosure, the second rotation link 120 may be located behind the first rotation link 110, and the fourth rotation link 140 and the fifth rotation link 150 may be located in front of the first rotation link 110.

The third rotation joint 163 connecting the second rotation link 120 and the third rotation link 130 may be installed at the uppermost one of the rotation links.

The sixth rotation joint 166 by which the fourth rotation link 140 and the fifth rotation link 150 are connected to each other may be located below the fourth rotation joint 164 by which the first rotation link 110 and the fourth rotation link 140 are connected to each other.

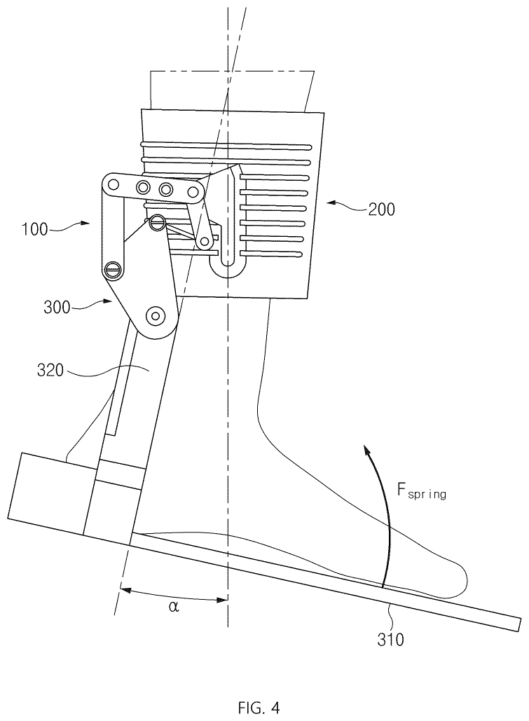

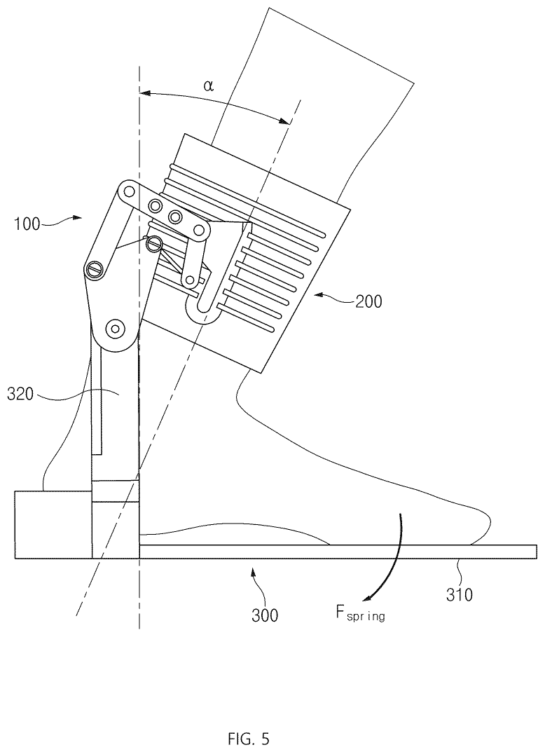

FIG. 4 is a side view illustrating an operation state in a state in which there is no foot drop of a wearable robot according to an embodiment of the present disclosure. FIG. 5 is a side view illustrating an operation state in a state in which a wearable robot is in a ground pushing-off state according to an embodiment of the present disclosure.

As illustrated in the accompanying drawings, when a walking cycle is analyzed based on a left foot of the user, the walking cycle may be divided into the following two operations.

A process, in which a rear end of a plantar surface of the left foot, i.e., a heel, comes into contact with the ground at the first time, a right foot then moves forward, and a tip of the plantar surface of the left foot is separated from the ground again, occupies 60% of the entire walking cycle. This can be referred to as a stance phase, and thereafter, a process in which the left foot moves forward and the rear end of the plantar surface then comes into contact with the ground again can be referred to as a swing phase.

FIGS. 4 and 5 relate to a method of controlling a wearable robot, and a neutral angle “\hat{α}” between the fifth rotation link 150 and the ground in a state in which the support part 300 of the wearable robot is in contact with the ground may be 5°≤\hat{α}≤12°. Here, E_{α}|_{α=\hat{α}}=0.

Therefore, when the neutral angle “\hat{α}” is greater than the above angle range, it can have a significant effect in preventing the foot drop, and when the neutral angle “\hat{α}” is smaller than the above angle range, a significant ground pushing-off effect may occur.

To describe this in detail, FIG. 4 illustrates a swing phase in which the advanced foot moves without contact with the ground during walking.

When an angle “α” is smaller than the neutral angle “\hat{α}”, an elastic force of the first elastic portion 171 may be calculated by the following equation:

$${F}_{spring} = - \frac{{dE}_{α}}{d α} .$$

That is, upward rotation of the support part 300 can occur in the swing phase.

The swing phase can be a phase in which the plantar surface of the user is swung while being spaced apart from the ground. When the user performs the swing phase, a relatively large force is not necessarily required, and a foot drop due to the elastic force F_{spring} of the first elastic portion 171 may be prevented.

FIG. 5 illustrates a stance phase in which the advanced foot is in contact with the ground during the walking.

When an angle “α” is greater than the neutral angle “\hat{α}”, the elastic force of the first elastic portion 171 may be calculated by the following equation:

$${F}_{spring} = - \frac{{dE}_{α}}{d α} .$$

That is, downward rotation of the support part 300 can occur in the stance phase.

The stance phase can be a state in which the plantar surface of the user is in contact with the ground, and in this phase, the ankle joint of the user should support a weight of the user. Further, in order for the user to move forward, the driving force should be generated by rotating the ankle joint while the ankle of the human body of the user supports the weight. That is, the ground pushing-off due to the elastic force F_{spring} of the first elastic portion 171 may be exhibited as well as a center of gravity that supports the ankle of the user may be correctly aligned, and thus standing stability may be secured.

Therefore, in the stance phase, the weight can be concentrated not only on a thigh and a lower leg of a pedestrian but also on an ankle of the pedestrian and more torque can be required as compared to the swing phase.

In this way, the walking of the pedestrian can be assisted according to a change in an angle between the connection part 200 and the support part 300 using the elastic force of the first elastic portion 171.

In particular, it may be identified that the walking assistance can be achieved by intensively transmitting the driving force during the stance phase in which a large force is applied to the ankle joint of the human body of the user due to bidirectional force transmission characteristics that can assist the walking.

In this way, the walking may be assisted so that the pedestrian does not have difficulty in the walking due to the force transmission characteristics of the first elastic portion 171 between the mechanism part 100 and the support part 300.

Thus, according to an embodiment of the present disclosure, misalignment between the ankle joint and the wearable robot may be resolved by simulating the nonlinear movement of the ankle joint of the human body using a linkage mechanism.

In an embodiment of the present disclosure, the mechanism part 100 including the rotation link parts 110 to 150 and the elastic portions 171 to 173 may be added in addition to the connection part 200 and the support part 300, the degree of freedom allowing the ankle joint to freely move according to an operation intention of the user may be secured, and thus an assistive force of the walking may be secured.

In an embodiment of the present disclosure, the wearable robot may be customized and thus convenient and may assist bidirectional movement of the joint of the human body, such as the foot drop or the ground pushing-off, during the walking.

According to a wearable robot according to an embodiment of the present disclosure, misalignment between an ankle joint and the wearable robot may be resolved by simulating nonlinear movement of the ankle joint of a human body using a linkage mechanism.

In an embodiment of the present disclosure, a degree of freedom allowing the ankle joint to freely move according to an operation intention of the user may be secured, and thus an assistive force of the walking may be secured.

In an embodiment of the present disclosure, nonlinear body joint movement may be assisted by adding not only a connection part and a support part, but also a mechanism part including a rotation link part and an elastic portion.

In an embodiment of the present disclosure, the wearable robot may be customized and thus convenient and may assist bidirectional movement of an ankle joint of a human body, such as foot drop or ground pushing-off, during walking.

The above description of example embodiments of the present disclosure is for illustration, and those skilled in the art to which the present disclosure pertains may understand that the present disclosure may be easily modified into other specific forms without changing the technical spirit or essential features of the present disclosure. Therefore, it can be understood that the example embodiments described above are illustrative but not necessarily limiting. The scopes of the present disclosure can be indicated by the appended claims, and all changes or modifications derived from the meaning and scopes of the appended claims and equivalents thereof can be construed as being included in the scopes of the present disclosure.

What is claimed is:

1. A wearable robot comprising:
a connection part configured to accommodate a lower leg of a human body of a user;
a mechanism part comprising:
a five-joint rotation link part coupled to the connection part, and
an elastic portion coupled to a partial rotation center of the five-joint rotation link part and configured to provide an elastic force to the connection part; and
a support part coupled to the mechanism part by a rotation joint,
wherein the wearable robot is configured such that in a process of synchronizing an ankle joint of the user and the mechanism part with each other by the connection part, one degree of freedom of underactuation among two degrees of freedom of the mechanism part can be offset and misalignment between the ankle joint and the mechanism part can be self-aligned.

2. The wearable robot of claim 1, wherein the five-joint rotation link part comprises:
a first rotation link connected to the support part by a first rotation joint;
a second rotation link connected to the first rotation link by a second rotation joint;
a third rotation link connected to the second rotation link by a third rotation joint;
a fourth rotation link connected to the first rotation link by a fourth rotation joint; and
a fifth rotation link connected to the third rotation link by a fifth rotation joint.

3. The wearable robot of claim 2, wherein the fifth rotation link is connected to the fourth rotation link by a sixth rotation joint.

4. The wearable robot of claim 3, wherein the sixth rotation joint is positioned below the fourth rotation joint by which the first rotation link and the fourth rotation link are connected to each other.

5. The wearable robot of claim 2, wherein the first rotation link is connected to the support part by the first rotation joint, wherein the first rotation link is connected to the second rotation link by the second rotation joint, and wherein the first rotation link is connected to the fourth rotation link by the fourth rotation joint.

6. The wearable robot of claim 2, wherein the elastic portion comprises:
a first elastic portion elastically supported by the first rotation joint of the first rotation link and configured to bias rotation of the first rotation link toward a front side about the first rotation joint;
a second elastic portion elastically supported by the second rotation joint of the first rotation link and configured to bias rotation of the second rotation link toward the front side about the second rotation joint; and
a third elastic portion elastically supported by the third rotation joint of the third rotation link and configured to bias rotation of the third rotation link toward the front side about the third rotation joint.

7. The wearable robot of claim 6, wherein the second elastic portion and the third elastic portion have a maximum deformation angle within a range of ±10°.

8. The wearable robot of claim 2, wherein the first rotation link is installed to support the support part in one degree of freedom of rotation and configured to be positioned behind the ankle joint.

9. The wearable robot of claim 2, wherein the second rotation link is positioned behind the first rotation link, and wherein the fourth rotation link and the fifth rotation link are positioned in front of the first rotation link.

10. The wearable robot of claim 2, wherein the third rotation joint connecting the second rotation link and the third rotation link is installed at an uppermost position relative to the first rotation link, the second rotation link, the third rotation link, the fourth rotation link, and the fifth rotation link.

11. The wearable robot of claim 2, wherein the fourth rotation link corresponds to face the third rotation link and is positioned below the third rotation link.

12. The wearable robot of claim 2, wherein the connection part is connected to the mechanism part by the fifth rotation link.

13. The wearable robot of claim 2, wherein the support part comprises:
a support frame configured to support a foot sole of the user; and
a support link connected between the first rotation link of the mechanism part and the support frame.

14. A wearable robot comprising:
a connection part configured to accommodate a lower leg of a human body of a user;
a support part configured to support a foot of the user; and
a mechanism part coupled to the connection part and the support part, wherein the mechanism part comprises:
a first rotation link connected to the support part by a first rotation joint,
a second rotation link connected to the first rotation link by a second rotation joint,
a third rotation link connected to the second rotation link by a third rotation joint,
a fourth rotation link connected to the first rotation link by a fourth rotation joint,
a fifth rotation link connected to the third rotation link by a fifth rotation joint, wherein the fifth rotation link is connected to the fourth rotation link by a sixth rotation joint,
a first elastic portion elastically supported by the first rotation joint of the first rotation link and configured to bias rotation of the first rotation link toward a front side of the wearable robot about the first rotation joint,
a second elastic portion elastically supported by the second rotation joint of the first rotation link and configured to bias rotation of the second rotation link toward the front side of the wearable robot about the second rotation joint, and
a third elastic portion elastically supported by the third rotation joint of the third rotation link and configured to bias rotation of the third rotation link toward the front side of the wearable robot about the third rotation joint.

15. The wearable robot of claim 14, wherein the mechanism part is coupled to the connection part at the sixth rotation joint.

16. The wearable robot of claim 14, wherein the sixth rotation joint is positioned below the fourth rotation joint by which the first rotation link and the fourth rotation link are connected to each other.

17. The wearable robot of claim 14, wherein the second elastic portion and the third elastic portion have a maximum deformation angle within a range of ±10°.

18. The wearable robot of claim 14, wherein the second rotation link is positioned behind the first rotation link, and wherein the fourth rotation link and the fifth rotation link are positioned in front of the first rotation link.

19. The wearable robot of claim 14, wherein the third rotation joint connecting the second rotation link and the third rotation link is installed at an uppermost position relative to the first rotation link, the second rotation link, the third rotation link, the fourth rotation link, and the fifth rotation link, and wherein the fourth rotation link corresponds to face the third rotation link and is positioned below the third rotation link.

20. A wearable robot comprising:
a leg connection part;
a foot support part; and
a mechanism part coupled to the leg connection part and the foot support part, wherein the mechanism part comprises:
a first rotation link connected to the foot support part by a first rotation joint,
a second rotation link connected to the first rotation link by a second rotation joint,
a third rotation link connected to the second rotation link by a third rotation joint,
a fourth rotation link connected to the first rotation link by a fourth rotation joint,
a fifth rotation link connected to the third rotation link by a fifth rotation joint,
wherein the fifth rotation link is connected to the fourth rotation link by a sixth rotation joint,
wherein the mechanism part is coupled to the leg connection part at the sixth rotation joint,
wherein the sixth rotation joint is positioned below the fourth rotation joint by which the first rotation link and the fourth rotation link are connected to each other,
wherein the second rotation link is positioned behind the first rotation link, and wherein the fourth rotation link and the fifth rotation link are positioned in front of the first rotation link,
wherein the third rotation joint connecting the second rotation link and the third rotation link is installed at an uppermost position relative to the first rotation link, the second rotation link, the third rotation link, the fourth rotation link, and the fifth rotation link, and
wherein the fourth rotation link corresponds to face the third rotation link and is positioned below the third rotation link,
a first elastic portion elastically supported by the first rotation joint of the first rotation link and configured to bias rotation of the first rotation link toward a front side of the wearable robot about the first rotation joint,
a second elastic portion elastically supported by the second rotation joint of the first rotation link and configured to bias rotation of the second rotation link toward the front side of the wearable robot about the second rotation joint, and
a third elastic portion elastically supported by the third rotation joint of the third rotation link and configured to bias rotation of the third rotation link toward the front side of the wearable robot about the third rotation joint.
